(19) United States
(12) Patent Application Publication
(10) Pub. No.: US 2013/0023442 A1
(43) Pub. Date: Jan. 24, 2013

(54) SINGLE NUCLEOTIDE POLYMORPHISM FOR PREDICTING RECURRENCE OF HEPATOCELLULAR CARCINOMA

(71) Applicants: Young Hwa Chung, Seoul (KR); Eun Sil Yu, Seoul (KR); Young-Joo Lee, Seoul (KR); Jeong A. Kim, Seoul (KR); Jong-Eun Lee, Seoul (KR)

(72) Inventors: Young Hwa Chung, Seoul (KR); Eun Sil Yu, Seoul (KR); Young-Joo Lee, Seoul (KR); Jeong A. Kim, Seoul (KR); Jong-Eun Lee, Seoul (KR)

(21) Appl. No.: 13/636,806

(22) PCT Filed: Mar. 22, 2011
(86) PCT No.: PCT/KR2011/001965

§ 371 Date: Sep. 24, 2012

(30) Foreign Application Priority Data
Mar. 22, 2010 (KR) 1020100025372
Mar. 22, 2011 (KR) 1020110025175

(51) Int. Cl. C40B 30/04; C12Q 1/68; C07H 21/00

(52) U.S. Cl. 506/9; 536/23.1; 435/6.11

(57) ABSTRACT

Single nucleotide polymorphisms (SNP) for predicting recurrence of hepatocellular carcinoma after curative surgical resection are provided. The SNPs have a significant correlation with higher risk of hepatocellular carcinoma recurrence after curative surgical resection. Therefore, the SNPs can be used in developing micro-arrays or test kits for predicting recurrence of hepatocellular carcinoma, and in screening drugs to prevent recurrence of hepatocellular carcinoma after curative surgical resection.

### TECHNICAL FIELD

The present invention relates to a single nucleotide polymorphism (SNP) that is useful to predict recurrence after hepatocellular carcinoma surgery, a micro-array or a test kit for predicting recurrence of hepatocellular carcinoma using the same, and a method for screening a drug for reducing recurrence of hepatocellular carcinoma.

### BACKGROUND ART

Hepatocellular carcinoma (HCC) is the most common and serious cancer to be in third place among all of malignant tumors from the viewpoint of cancer development and deaths in this country. The hepatocellular carcinoma is one of the most hypervascular tumors.

The most effective modality to treat HCC is surgical resection or liver transplantation. However, only 10% to 20% of the patients with hepatocellular carcinoma can be operated due to the size and number of the tumor that cannot be removed, a bad liver function, all kinds of intrahepatic or extrahepatic metastases, and the like. Even with regard to the operable patients with hepatocellular carcinoma, frequent postoperative recurrence is a main limiting factor in survival for a long time period.

The development and rapid progression of hepatocellular carcinoma involve various mechanisms. Among them, the promotion of angiogenesis by hypoxia plays very important roles. In addition, under the hypoxic environment in liver, metastatic tumor antigen 1 (MTA1) contributes to angiogenesis of malignant tumor by enhancing the expression of a vascular endothelial growth factor (VEGF) by structurally stabilizing hypoxia inducible factor 1 (HIF1) (Moon E J, et al., 2004; Moon E J, et al., 2006).

Meanwhile, after infection of hepatitis B virus, about 5% to 10% becomes chronic hepatitis B, and some of them may progress to cirrhosis or hepatocellular carcinoma. Like this, it is understood that various clinical progressions exhibited after the infection of hepatitis B virus depend on a difference of each individual's genetic predisposition as well as a difference of virus itself.

Genetic predisposition means that there is a tiny difference in various genes between individuals. Recently, the level of the difference has been reported from individual to individual through genome research. Among genetic variation, single nucleotide polymorphisms (SNPs) have been known to change the gene functions, and 710,000 polymorphisms in approximately 11 million SNPs have been reported in human genome so far (NCBI, dbSNP).

Recently, so many researches are being actively carried out to determine whether a tiny change in the base sequence actually influences susceptibilities to a certain disease, and to find the genetic variations predisposing the disease (Ludwig J A, and Weinstein J N, 2005; Suh Y, and Vijg J, 2005; Chanock S, 2001).

### DISCLOSURE

### Technical Problem

In order to address the above problems in the conventional technologies, the inventors found SNP exhibiting a meaningful correlation with recurrence after hepatocellular carcinoma surgery. Accordingly, the present invention was completed.

Accordingly, an object of the present invention is to provide a single nucleotide polymorphism (SNP) that is useful to predict recurrence after hepatocellular carcinoma surgery.

In addition, another object of the present invention is to provide a micro-array for predicting recurrence of hepatocellular carcinoma using the single nucleotide polymorphism (SNP).

In addition, still another object of the present invention is to provide a test kit for predicting recurrence of hepatocellular carcinoma.

In addition, still another object of the present invention is to provide a method for predicting recurrence of hepatocellular carcinoma using the sing nucleotide polymorphism (SNP).

In addition, still another object of the present invention is to provide a method for screening a drug for preventing recurrence of hepatocellular carcinoma using the single nucleotide polymorphism (SNP).

### Technical Solution

In order to achieve the above objects, an exemplary embodiment of the present invention provides a single nucleotide polymorphism (SNP) for predicting recurrence of hepatocellular carcinoma, in which the SNP includes at least one polynucleotide selected from the group consisting of C allele (CC or GC genotype) in −291C/G (rs3213221) [SEQ ID NO. 1] of IGF2 gene; T allele (CT or TT genotype) in −13021C/T (rs3741208) [SEQ ID NO. 2] of IGF2 gene; T allele (CT or TT genotype) in 66378C/T (rs1048201) [SEQ ID NO. 3] of FGF2 gene; G allele (GG or GA genotype) in 50012A/G (rs6534367) [SEQ ID NO. 4] of FGF2 gene; GC haplotype in 6310 (rs2585) [SEQ ID NO. 5]/4702 (rs3802971) [SEQ ID NO. 6] of IGF2 gene; homozygotic CC haplotype in −11228 (rs2239681) [SEQ ID NO. 7]/−13021 (rs3741208) [SEQ ID NO. 2] of IGF2 gene; and CT haplotype in −11228 (rs2239681) [SEQ ID NO. 7]/−13021 (rs3741208) [SEQ ID NO. 2] of IGF2 gene; or a complementary nucleotide thereof.

The recurrence of hepatocellular carcinoma may be related to recurrence of hepatocellular carcinoma after curative surgical resection in patients with HCC treated with curative surgical resection.

In addition, an exemplary embodiment of the present invention provides a micro-array for predicting recurrence of hepatocellular carcinoma, in which the micro-array includes the polynucleotide of the single nucleotide polymorphism (SNP) for predicting recurrence of hepatocellular carcinoma after curative surgical resection, a polypeptide encoded by the same, or cDNA thereof.

In addition, an exemplary embodiment of the present invention provides a test kit for predicting recurrence of hepatocellular carcinoma, including the micro-array.

The test kit according to the present invention may further include a primer set that is used for isolating and amplifying DNA including a relevant SNP from a clinical specimen in addition to a micro-array of the present invention.

In addition, an embodiment of the present invention provides a test kit for predicting recurrence of hepatocellular carcinoma, using a single-base extension (SBE) reaction in order for genotyping SNP.

The test kit for predicting recurrence of hepatocellular carcinoma using the single-base extension reaction is designed to confirm whether C allele (CC or GC genotype) in −291C/G (rs3213221) [SEQ ID NO. 1] of IGF2 gene; T allele (CT or TT genotype) in −13021C/T (rs3741208) [SEQ ID NO. 2] of IGF2 gene; homozygotic GC haplotype in 6310 (rs2585) [SEQ ID NO. 5]/4702 (rs3802971) [SEQ ID NO. 6] of IGF2 gene; and CT haplotype in −11228 (rs2239681) [SEQ ID NO. 7]/−13021 (rs3741208) [SEQ ID NO. 2] of IGF2 gene exist.

An embodiment of the present invention provides a test kit for predicting recurrence of hepatocellular carcinoma, using a single-base extension reaction, including a forward primer for amplifying −13021 (rs3741208) region of IGF2 gene; a reverse primer for amplifying −13021 (rs3741208) of IGF2 gene; a primer for genotyping −13021 (rs3741208) region of IGF2 gene; a forward primer for amplifying 6310 (rs2585) region of IGF2 gene; a reverse primer for amplifying 6310 (rs2585) region of IGF2 gene; a primer for genotyping 6310 (rs2585) region of IGF2 gene; a forward primer for amplifying −11228 (rs2239681) region of IGF2 gene; a reverse primer for amplifying −11228 (rs2239681) region of IGF2 gene; a primer for genotyping −11228 (rs2239681) region of IGF2 gene; a forward primer for amplifying 4702 (rs3802971) region of IGF2 gene; a reverse primer for amplifying 4702 (rs3802971) region of IGF2 gene; a primer for genotyping 4702 (rs3802971) region of IGF2 gene; a forward primer for amplifying −291C/G (rs3213221) region of IGF2 gene; a reverse primer for amplifying −291C/G (rs3213221) region of IGF2 gene; and a primer for genotyping −291C/G (rs3213221) region of IGF2 gene.

According an embodiment, in the test kit for predicting recurrence of hepatocellular carcinoma, the forward primer for amplifying −13021 (rs3741208) region of IGF2 gene may be a primer of SEQ ID NO. 17; the reverse primer for amplifying −13021 (rs3741208) region of IGF2 gene may be a primer of SEQ ID NO. 18; the primer for genotyping −13021 (rs3741208) region of IGF2 gene may be a primer of SEQ ID NO. 35; the forward primer for amplifying 6310 (rs2585) region of IGF2 gene may be a primer of SEQ ID NO. 20; the reverse primer for amplifying 6310 (rs2585) region of IGF2 gene may be a primer of SEQ ID NO. 21; the primer for genotyping 6310 (rs2585) region of IGF2 gene may be a primer of SEQ ID NO. 36; the forward primer for amplifying −11228 (rs2239681) region of IGF2 gene may be a primer of SEQ ID NO. 37; the reverse primer for amplifying −11228 (rs2239681) region of IGF2 gene may be a primer of SEQ ID NO. 38; the primer for genotyping −11228 (rs2239681) region of IGF2 gene may be a primer of SEQ ID NO. 39; the forward primer for amplifying 4702 (rs3802971) region of IGF2 gene may be a primer of SEQ ID NO. 40; the reverse primer for amplifying 4702 (rs3802971) region of IGF2 gene may be a primer of SEQ ID NO. 41; the primer for genotyping 4702 (rs3802971) region of IGF2 gene may be a primer of SEQ ID NO. 42; the forward primer for amplifying −291C/G (rs3213221) region of IGF2 gene may be a primer of SEQ ID NO. 43; the reverse primer for amplifying −291C/G (rs3213221) region of IGF2 gene may be a primer of SEQ ID NO. 44; and the primer for genotyping −291C/G (rs3213221) region of IGF2 gene may be a primer of SEQ ID NO. 45.

In addition, the present invention provides a method for predicting recurrence of hepatocellular carcinoma, the method including a step of obtaining a nucleic acid sample from a clinical specimen; and a step of determining a nucleotide sequence of at least any one polymorphism regions of at least one polynucleotide selected from the group consisting of C allele (CC or GC genotype) in −291C/G (rs3213221) of IGF2 gene; T allele (CT or TT genotype) in −13021C/T (rs3741208) of IGF2 gene; T allele (CT or TT genotype) in 66378C/T (rs1048201) of FGF2 gene; G allele (GG or GA genotype) in 50012A/G (rs6534367) of FGF2 gene; GC haplotype in 6310 (rs2585)/4702 (rs3802971) of IGF2 gene; homozygotic CC haplotype in −11228 (rs2239681)/−13021 (rs3741208) of IGF2 gene; and CT haplotype in −11228 (rs2239681)/−13021 (rs3741208) of IGF2 gene; or a complementary nucleotide thereof.

The step of determining the nucleotide sequence of the polymorphism region may include a step of hybridizing the nucleic acid sample to a micro-array fixed with the polynucleotide or the complementary nucleotide thereof and a step of detecting a hybridization result thus obtained.

In addition, the present invention provides a method for screening a drug for preventing recurrence of hepatocellular carcinoma, the method including a step of contacting a polypeptide encoded by the polynucleotide or the complementary nucleotide thereof of the single nucleotide polymorphism (SNP) for predicting recurrence of hepatocellular carcinoma with a candidate material; and a step of determining whether the candidate material has activity to enhance or inhibit a function of the polypeptide.

According to the present invention, a micro-array or a test kit for predicting recurrence of hepatocellular carcinoma can be developed by using a single nucleotide polymorphism (SNP) that is useful for predicting recurrence after the operation of hepatocellular carcinoma, and the recurrence after the operation of hepatocellular carcinoma can be prevented by screening a drug for reducing recurrence of hepatocellular carcinoma.

### DESCRIPTION OF DRAWINGS

FIG. 1 shows a cumulative recurrence rate of hepatocellular carcinoma according to genotype at −291C/G (rs3213221) location of IGF2 gene;

FIG. 2 shows a cumulative recurrence rate of hepatocellular carcinoma according to genotype at −13021C/T (rs3741208) location of IGF2 gene;

FIG. 3 shows a cumulative recurrence rate of hepatocellular carcinoma according to GC haplotype of 6310 (rs2585)/4702 (rs3802971) of IGF2 gene;

FIG. 4 shows a cumulative recurrence rate of hepatocellular carcinoma according to CC haplotype of −11228 (rs2239681)/−13021 (rs3741208) of IGF2 gene;

FIG. 5 shows a cumulative recurrence rate of hepatocellular carcinoma according to CT haplotype of −11228 (rs2239681)/−13021 (rs3741208) of IGF2 gene; and

FIGS. 6 to 8 show results of genotyping using a test kit for predicting recurrence of hepatocellular carcinoma according to an embodiment of the present invention.

In order to achieve the above objects, the present invention provides single nucleotide polymorphisms (SNP) for predicting recurrence of hepatocellular carcinoma, the SNPs including at least one polynucleotide selected from the group consisting of C allele (CC or GC genotype) in −291C/G (rs3213221) [SEQ ID NO. 1] of IGF2 gene; T allele (CT or TT genotype) in −13021C/T (rs3741208) [SEQ ID NO. 2] of IGF2 gene; T allele (CT or TT genotype) in 66378C/T (rs1048201) [SEQ ID NO. 3] of FGF2 gene; G allele (GG or GA genotype) in 50012A/G (rs6534367) [SEQ ID NO. 4] of FGF2 gene; GC haplotype in 6310 (rs2585) [SEQ ID NO. 5]/4702 (rs3802971) [SEQ ID NO. 6] of IGF2 gene; homozygotic CC haplotype in −11228 (rs2239681) [SEQ ID NO. 7]/−13021 (rs3741208) [SEQ ID NO. 2] of IGF2 gene; and CT haplotype in −11228 (rs2239681) [SEQ ID NO. 7]/−13021 (rs3741208) [SEQ ID NO. 2] of IGF2 gene; or a complementary nucleotide thereof.

In addition, the recurrence of hepatocellular carcinoma may be related to recurrence of hepatocellular carcinoma in patients with hepatocellular carcinoma treated with curative surgical resection.

In addition, the present invention provides a micro-array for predicting recurrence of hepatocellular carcinoma, the micro-array including the polynucleotide of the single nucleotide polymorphisms (SNP) for predicting the recurrence of hepatocellular carcinoma, a polypeptide encoded by the same, or cDNA thereof.

The micro-array for predicting the recurrence of hepatocellular carcinoma may be manufactured by the general method known by a person of ordinary skill in the art, and for example, the polynucleotide that is included in the micro-array for diagnosing the recurrence of hepatocellular carcinoma may be fixed to a substrate coated with an active group selected from the group consisting of an amino-silane, a poly-L-lysine, and aldehyde, and the substrate may be selected from the group consisting of a silicon wafer, glass, quartz, metal, and plastic. The method for fixing the polynucleotide to the substrate may include a micropipetting method using a piezoelectric way, a method suing a spotter of a pin type, and the like.

In addition, the present invention provides a test kit for predicting recurrence of hepatocellular carcinoma, the test kit including the micro-array.

The test kit according to the present invention may further include a set of primers that is used for isolating and amplifying DNA including a relevant SNP from a clinical specimen in addition to the micro-array of the present invention. The appropriate set of primers may be easily designed by a person of ordinary skill in the art with reference to the sequences of the present invention.

In addition, an embodiment of the present invention provides a test kit for predicting recurrence of hepatocellular carcinoma using a single-base extension (SBE) reaction in order for genotyping SNP. In this case, the primers for an amplification (Forward direction and Reverse direction) and extension (Genotyping) should be designed for the single-base extension (SBE).

The test kit for predicting recurrence of hepatocellular carcinoma using the single-base extension reaction may be a test kit for analyzing a genotype of SNaPshot method.

The test kit for predicting recurrence of hepatocellular carcinoma using the single-base extension reaction is designed in order to confirm whether C allele (CC or GC genotype) in −291C/G (rs3213221) [SEQ ID NO. 1] of IGF2 gene; T allele (CT or TT genotype) in −13021C/T (rs3741208) [SEQ ID NO. 2] of IGF2 gene; homozygotic GC haplotype in 6310 (rs2585) [SEQ ID NO. 5]/4702 (rs3802971) [SEQ ID NO. 6] of IGF2 gene; and CT haplotype in −11228 (rs2239681) [SEQ ID NO. 7]/−13021 (rs3741208) [SEQ ID NO. 2] of IGF2 gene exist.

An embodiment of the present invention provides a test kit for predicting recurrence of hepatocellular carcinoma, the test kit including a forward primer for amplifying −13021 (rs3741208) region of IGF2 gene; a reverse primer for amplifying −13021 (rs3741208) region of IGF2 gene; a primer for genotyping −13021 (rs3741208) region of IGF2 gene; a forward primer for amplifying 6310 (rs2585) region of IGF2 gene; a reverse primer for amplifying 6310 (rs2585) region of IGF2 gene; a genotyping primer for amplifying 6310 (rs2585) region of IGF2 gene; a forward primer for amplifying −11228 (rs2239681) region of IGF2 gene; a reverse primer for amplifying −11228 (rs2239681) region of IGF2 gene; a primer for genotyping −11228 (rs2239681) region of IGF2 gene; a forward primer for amplifying 4702 (rs3802971) region of IGF2 gene; a reverse primer for amplifying 4702 (rs3802971) region of IGF2 gene; a primer for genotyping 4702 (rs3802971) region of IGF2 gene; a forward primer for amplifying −291C/G (rs3213221) region of IGF2 gene; a reverse primer for amplifying −291C/G (rs3213221) region of IGF2 gene; and a primer for genotyping −291C/G (rs3213221) region of IGF2 gene, and using a single-base extension reaction.

According to an embodiment, in the test kit for predicting the recurrence of hepatocellular carcinoma, the forward primer for amplifying −13021 (rs3741208) region of IGF2 gene may be a primer of SEQ ID NO. 17; the reverse primer for amplifying −13021 (rs3741208) of IGF2 gene may be a primer of SEQ ID NO. 18; the primer for genotyping −13021 (rs3741208) region of IGF2 gene may be a primer of SEQ ID NO. 35; the forward primer for amplifying 6310 (rs2585) region of IGF2 gene may be a primer of SEQ ID NO. 20; the reverse primer for amplifying 6310 (rs2585) region of IGF2 gene may be a primer of SEQ ID NO. 21; the genotyping primer for amplifying 6310 (rs2585) region of IGF2 gene may be a primer of SEQ ID NO. 36; the forward primer for amplifying −11228 (rs2239681) region of IGF2 gene may be a primer of SEQ ID NO. 37; the reverse primer for amplifying −11228 (rs2239681) region of IGF2 gene may be a primer of SEQ ID NO. 38; the primer for genotyping −11228 (rs2239681) region of IGF2 gene may be a primer of SEQ ID NO. 39; the forward primer for amplifying 4702 (rs3802971) region of IGF2 gene may be a primer of SEQ ID NO. 40; the reverse primer for amplifying 4702 (rs3802971) region of IGF2 gene may be a primer of SEQ ID NO. 41; the primer for genotyping 4702 (rs3802971) region of IGF2 gene may be a primer of SEQ ID NO. 42; the forward primer for amplifying −291C/G (rs3213221) region of IGF2 gene may be a primer of SEQ ID NO. 43; the reverse primer for amplifying −291C/G (rs3213221) region of IGF2 gene may be a primer of SEQ ID NO. 44; and the primer for genotyping −291C/G (rs3213221) region of IGF2 gene may be a primer of SEQ ID NO. 45.

In addition, the present invention provides a method for predicting recurrence of hepatocellular carcinoma, the method including a step of obtaining a nucleic acid sample from a clinical specimen; and a step of determining a nucleotide sequence of at least any one polymorphism region of at least one polynucleotide selected from the group consisting of C allele (CC or GC genotype) in −291C/G (rs3213221) of IGF2 gene; T allele (CT or TT genotype) in −13021C/T (rs3741208) of IGF2 gene; T allele (CT or TT genotype) in 66378C/T (rs1048201) of FGF2 gene; G allele (GG or GA genotype) in 50012A/G (rs6534367) of FGF2 gene; GC haplotype in 6310 (rs2585)/4702 (rs3802971) of IGF2 gene; homozygotic CC haplotype in −11228 (rs2239681)/−13021 (rs3741208) of IGF2 gene; and CT haplotype in −11228 (rs2239681)/−13021 (rs3741208) of IGF2 gene; or a complementary nucleotide thereof.

The nucleic acid may include DNA, mRNA, or cDNA synthesized from mRNA.

The step of determining the nucleotide sequence of the polymorphism region may include a step of hybridizing the nucleic acid sample to a micro-array fixed with the polynucleotide or the complementary nucleotide thereof, and a step of detecting the hybridization result thus obtained.

For example, DNA is isolated from a tissue, body fluid, or cell of objects; amplified through PCR; and then SNP is analyzed. The SNP analysis may be performed by using the known general method. For example, the SNP analysis may be performed by using a real time PCR system or by directly determining the nucleotide sequence of nucleic acid by a dideoxy method. Alternatively, the SNP analysis may be performed by determining the nucleotide sequence of polymorphism region by measuring the degree of hybridization obtained by hybridizing the DNA with a probe including the sequence of SNP region or a complementary probe thereof, or may be performed by using allele-specific probe hybridization, allele-specific amplification, sequencing, 5′ nuclease digestion, molecular beacon assay, oligonucleotide ligation assay, size analysis, single-stranded conformation polymorphism, and the like.

In addition, the present invention provides a method for screening a drug for preventing recurrence of hepatocellular carcinoma, the method including a step of contacting a polypeptide encoded by the polynucleotide of a single nucleotide polymorphism (SNP) for diagnosing recurrence of hepatocellular carcinoma, or the complementary nucleotide thereof with a candidate material; and a step of determining whether the candidate material has activity to improve or inhibit a function of the polypeptide.

In the screening method of the present invention, the reaction between the polypeptide and the candidate material may be determined by using general methods used for determining whether the reaction between protein-protein and the reaction between protein-compound are occurred or not. For example, there may be a method for measuring activity after reacting the protein and the candidate material, a yeast two-hybrid, a search of phage-displayed peptide clone bonded to the protein, a high throughput screening (HTS) using a natural substance, chemical library, and the like, a drug hit HTS, a cell-based screening, a method for screening using DNA array, or the like.

In the screening method of the present invention, the candidate material may be individual nucleic acids, proteins, other extracts, natural substances, compounds, or the like, that are assumed to have potential to be a diagnostic agent for recurrence of hepatocellular carcinoma or are randomly selected according to a general selection method.

### BEST MODE

Hereinafter, the present invention will be described in more detail with reference to the following Examples. However, the present invention is not limited to the following Examples.

### Example 1

### SNP Selection

A biallelic SNPs included in ±2 kb of angiogenesis-related genes, that is, VEGF, HIF1a, IGF2, FGF2, or MTA1, was subjected. As the positions of SNPs of the genes, 5′-nontranslation, promoter, exon, and gene loci regions were selected with reference to gene information (http://www.ncbi.nlm.gov/project/SNP) that is already known.

The IDs of subjected SNPs are as follows:

rs699947, rs25648, rs3025000, rs3025010, rs3025035, rs3025040, rs10434, rs998584, rs45533131/rs1957757, rs2301113, rs2057482/rs2585, rs3802971, rs3213221, rs3741212, rs2239681, rs3741208, rs1004446, rs7924316, rs3842748, rs2070762/rs308395, rs308428, rs11938826, rs17472986, rs308442, rs308379, rs308381, rs6534367, rs1048201, rs3747676/rs4983413, and DL1002505.

Among the above SNPs, SNPs having a haplotype frequency of equal to or greater than 5% were selected, and the haplotype frequencies were analyzed by using PHASE software v2.1. In addition, linkage disequilibrium was analyzed by using Haploview program v3.2 (http://www.broad.mit.edu/mpg/haploview/index.php).

### Example 2

### SNP Genotype Analysis

1. SNP Genotype Analysis

A primer set that can amplify the region including SNP of Example 1 and TaqMan probe including SNP region were manufactured by using primer express software. As the TaqMan probe, each of the probes that are suitable for wide type and mutant alleles was manufactured according to the sequence of SNP.

A probe was manufactured by tacking a fluorescent dye on one side of the TaqMan probe and tacking a quencher that can inhibit the color of the fluorescent dye on the other side. In this case, separate fluorescent dyes having different colors were tacked to the wild type and mutant alleles, respectively.

Three types of primers disclosed in the following Table 1 were mixed together, and then PCR reaction was performed by using the mixed primers to distinguish the mismatch of a pair of single nucleotide according to active property of exonuclease of Taq polymerase.

TABLE 1

| rs No. | Strand | Primer Sequence | |
|---|---|---|---|
| rs1048201 | Forward | Forward | ATGATATACATATCTGACTTCCCAA |
| | | | (SEQ ID NO. 8) |
| | | Reverse | AAGAGACTGGTATAAAATCAGAATTCA |
| | | | (SEQ ID NO. 9) |
| | | Genotyping | CGTGCCGCTCGTGATAGAATAGCTCCAGGATTTGTGTGCTGTTGC |
| | | | (SEQ ID NO. 10) |
| rs6534367 | Forward | Forward | TTCTTCTATTATGCARTTGTTTGAAG |
| | | | (SEQ ID NO. 11) |
| | | Reverse | TTACATTCTCAACTAGTGTTCTACATTG |
| | | | (SEQ ID NO. 12) |
| | | Genotyping | ACGCACGTCCACGGTGATTTATAAATRTATACAATTTTGATTATT |
| | | | (SEQ ID NO. 13) |
| rs308428 | Forward | Forward | GCATGTTTTGGGAACCAA |
| | | | (SEQ ID NO. 14) |
| | | Reverse | ATTAAAACCCTCCATTGACTCC |
| | | | (SEQ ID NO. 15) |
| | | Genotyping | CGTGCCGCTCGTGATAGAATGAAGTTTGGAATGGCAAGAAGTAAG |
| | | | (SEQ ID NO. 16) |
| rs3741208 | Reverse | Forward | acaggtaaagcttccttcc |
| | | | (SEQ ID NO. 17) |
| | | Reverse | gccatcaggaggagaga |
| | | | (SEQ ID NO. 18) |
| | | Genotyping | CgACTgTAggTgCgTAACTCgagggcVgttgttgcctctcccggY |
| | | | (SEQ ID NO. 19) |
| rs2585 | Forward | Forward | AATGTCACCTGTGCCTGC |
| | | | (SEQ ID NO. 20) |
| | | Reverse | TTAAAGACAAAACCCAAGCATG |
| | | | (SEQ ID NO. 21) |
| | | Genotyping | TGCGGCCCGTGTTTGACTYAACTCA |
| | | | (SEQ ID NO. 22) |
| DL1002505 | Forward | Forward | TGTCCGGCAGCAGGAGGA |
| | | | (SEQ ID NO. 23) |
| | | Reverse | ACGACACACTGCCAGACCA |
| | | | (SEQ ID NO. 24) |
| | | Genotyping | aggactgggcctcctgcgtgctggc |
| | | | (SEQ ID NO. 25) |
| rs308395 | Forward | Forward | gaggcacgtccatacttg |
| | | | (SEQ ID NO. 26) |
| | | Reverse | cagcgtctcacacactga |
| | | | (SEQ ID NO. 27) |
| | | Genotyping | ctcttctatggcctactttctactg |
| | | | (SEQ ID NO. 28) |
| rs11938826 | Forward | Forward | TTGGGGAAGGCTGATAAT |
| | | | (SEQ ID NO. 29) |
| | | Reverse | GCCATATTTCGGCTAACA |
| | | | (SEQ ID NO. 30) |
| | | Genotyping | CCCAGAAAAGAGGGTACTTCACACCAG |
| | | | (SEQ ID NO. 31) |
| rs3213221 | Reverse | Forward | taggacggaggccaggtc |
| | | | (SEQ ID NO. 32) |
| | | Reverse | aggtgcccctcccaaac |
| | | | (SEQ ID NO. 33) |
| | | Genotyping | aattttacacgagggKtgaccatct |
| | | | (SEQ ID NO. 34) |

A final SNP marker result was determined after verifying compatibility of the results that are read independently by more than two researchers. A result of individual SNP marker is represented by major allele homozygote, heterozygote, or minor allele homozygote according to a single nucleotide polymorphism allele. The results of the whole subjects were analyzed by the ratio of major and minor allele frequencies and frequencies of the three genotypes. The results were verified by Hardy-Weinberg equilibrium test.

Student's t-test and chi-squared test were used and odds ratio was calculated to analyze the correlation between MTA1 expressions in hepatocellular carcinoma tissue and SNPs.

2. Result

SNPs having a significant correlation with recurrence after curative surgical resection were found in IGF2 and FGF2 genes. In the case of having C allele (CC or GC genotype) (P=0.005) of −291 C/G (rs3213221) or T allele (CT or TT genotype) (P=0.003) of −13021 (rs3741208) in IGF2 gene, a recurrence rate was significantly high after curative surgical resection.

In the case of having T allele (CT or TT genotype) (P=0.044) of 66378 C/T (rs1048201) or G allele (GG or GA genotype) (P=0.019) of 50012 A/G (rs6534367) in FGF2 gene, a recurrence rate was significantly high after curative surgical resection.

In a haplotype analysis, a statistical significance was found only in IGF2 gene. Recurrence rate was significantly high in a patient having GC haplotype at 6310(rs2585)/4702(rs3802971) location of IGF2 gene (OR 1.653 (1.149-2.379), P=0.02). Particularly, a recurrence rate of hepatocellular carcinoma was significantly high in the patients having homozygotic GC haplotype as compared with patients without the homozygotic GC haplotype (OR 2.779 (1.205-6.40), P=0.04). In addition, CT haplotype at −11228 (rs2239681)/−13021 (rs3741208) location of IGF2 gene was related to a high recurrent rate after curative surgical resection (OR 1.88 (1.247-2.834), P=0.006), while the recurrent rate was significantly low in patients having homozygotic CC haplotype after curative surgical resection (OR 0.243 (0.07-0.841), P=0.049).

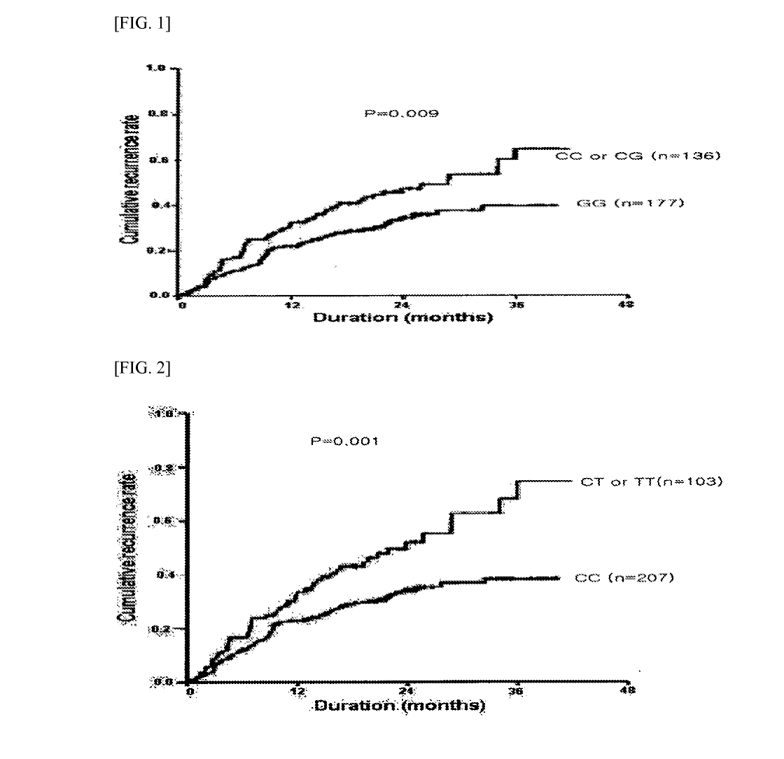

In addition, when comparing a cumulative recurrence rate after curative surgical resection according to each of SNPs, it could be found that in the case with C allele (CC or GC genotype) of −291 C/G (rs3213221) location in IGF2 gene, the cumulative recurrence rate was significantly high as compared with the rate of patients with GG genotype (P=0.009). As illustrated in FIG. 1, the 1-year, 2-year and 3-year cumulative recurrence rates of the patients with C allele (CC or GC genotype) were 32.5%, 46.9% and 64.4%, respectively, which are significantly higher than the rates of patients with GG genotype (22.0%, 34.5% and 19.6%, respectively). As illustrated in FIG. 2, in the cases having T allele (CT or TT genotype) at −13021 C/T (rs3741208) location of IGF2 gene, the cumulative recurrence rate was significantly high after curative surgical resection as compared with the rate of patients with CC genotype (CT or TT vs. CC; the 1-, 2- and 3-year cumulative recurrence rates were 33.5%, 52.0% and 74.4% vs. 22.9%, 34.2% and 38.4%, respectively; P=0.001).

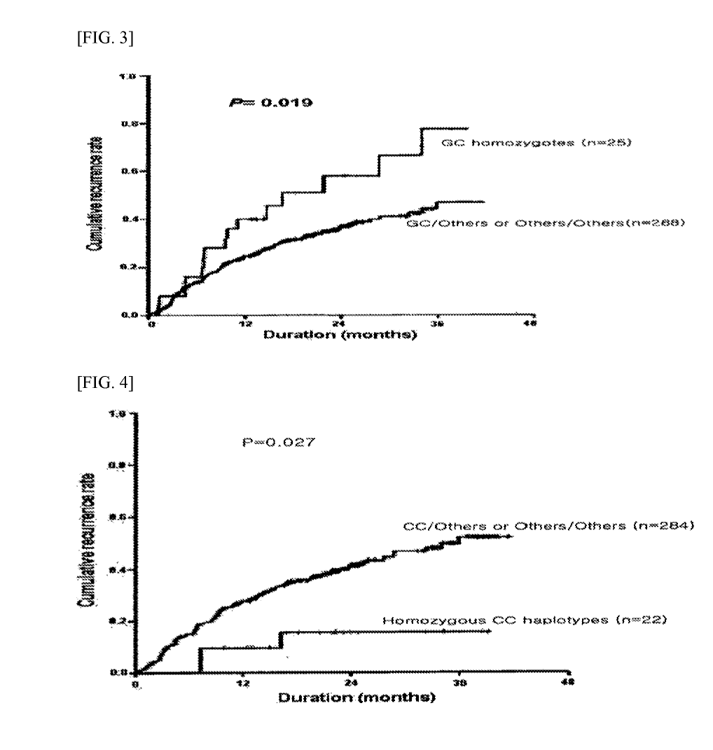

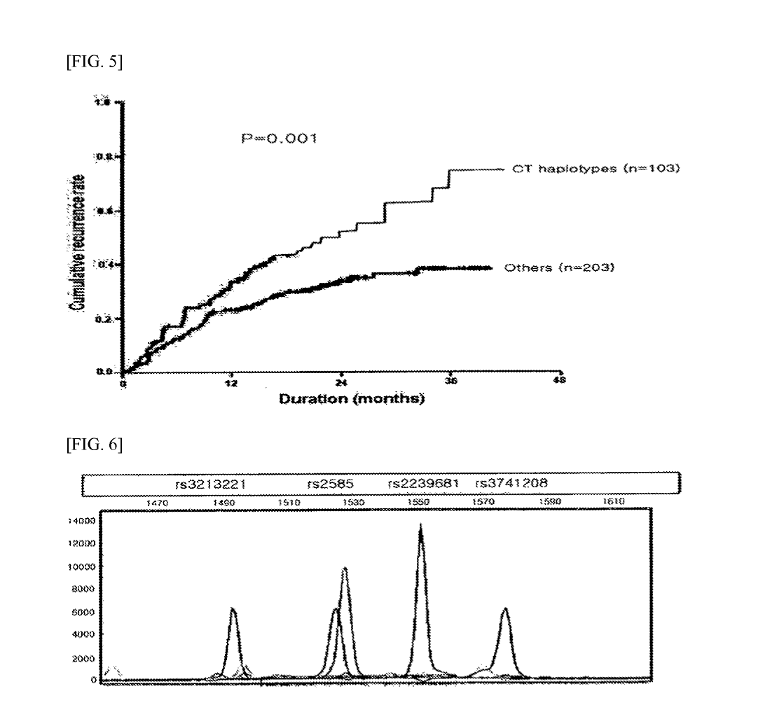

As illustrated in FIG. 3, in the cases having GC haplotype homozygote at 6310 (rs2585)/4702 (rs3802971) location of IGF2 gene, the cumulative recurrence rate was significantly high after curative surgical resection as compared with the rates of patients without GC haplotype or patients with heterozygote (P=0.019). In addition, CC haplotype and CT haplotype at −11228 (rs2239681)/−13021 (rs3741208) location of IGF2 gene had a significant relationship with the cumulative recurrence rate of HCC after curative surgical resection. In patients with −11228/−13021 CC homozygote, the cumulative recurrence rate was significantly low compared with the rates of the patients without it or patients with heterozygote (P=0.027) (see FIG. 4). In addition, as illustrated in FIG. 5, in patients with −11228/−13021 CT haplotype, the cumulative recurrence rate was significantly high as compared with the patients without CT haplotype (P=0.001).

### Example 3

### Test Kit for Predicting Recurrence of Hepatocellular Carcinoma and Method of Analyzing Single Nucleotide Polymorphism for Predicting Recurrence of Hepatocellular Carcinoma

The primer sequences for an amplification (Forward direction and Reverse direction) and extension (Genotyping) in genotyping single nucleotide polymorphisms of rs3741208, rs2585, rs2239681, rs3802971, and rs3213221, which are single nucleotide polymorphism regions that is useful for predicting recurrence after hepatocellular carcinoma surgery, are shown in the following Table 2. The following SEQ ID NO. 35 (5′-GCCTSCTGACCACCAGCAAGAAATTGGACAGGAGACYGARGAGAAA-3′) may be a primer set including SEQ ID NO. 46 (5′-GCCTGCTGACCACCAGCAAGAAATTGGACAGGAGACCGAAGAGAAA-3′), SEQ ID NO. 47 (5′-GCCTGCTGACCACCAGCAAGAAATTGGACAGGAGACTGAAGAGAAA-3′), SEQ ID NO. 48 (5′-GCCTCCTGACCACCAGCAAGAAATTGGACAGGAGACCGAAGAGAAA-3′), SEQ ID NO. 49 (5′-GCCTCCTGACCACCAGCAAGAAATTGGACAGGAGACTGAAGAGAAA-3′), SEQ ID NO. 50 (5′-GCCTGCTGACCACCAGCAAGAAATTGGACAGGAGACCGAGGAGAAA-3′), SEQ ID NO. 51 (5′-GCCTGCTGACCACCAGCAAGAAATTGGACAGGAGACTGAGGAGAAA-3′), SEQ ID NO. 52 (5′-GCCTCCTGACCACCAGCAAGAAATTGGACAGGAGACCGAGGAGAAA-3′), and SEQ ID NO. 53 (5′-GCCTCCTGACCACCAGCAAGAAATTGGACAGGAGACTGAGGAGAAA-3′) in approximately 1:1:1:1:1:1:1:1:1.

TABLE 2

| rs No. | Strand | Primer Sequence | |
|---|---|---|---|
| rs3741208 | Reverse | Forward | 5′-ACAGGTAAAGCTTCCTTCC-3′ |
| | | | (SEQ ID NO. 17) |
| | | Reverse | 5′-GCCATCAGGAGGAGAGA-3′ |
| | | | (SEQ ID NO. 18) |
| | | Genotyping | 5′-GCCTSCTGACCACCAGCAAGAAATTGGACAG |
| | | | GAGACYGARGAGAAA-3′ |
| | | | (SEQ ID NO. 35) |
| rs2585 | Forward | Forward | 5′-AATGTCACCTGTGCCTGC-3′ |
| | | | (SEQ ID NO. 20) |
| | | Reverse | 5′-TTAAAGACAAAACCCAAGCATG-3′ |
| | | | (SEQ ID NO. 21) |
| | | Genotyping | 5′-GGTCCMCCTTGCGGCCCGTGTTTGACTYAACTCA-3′ |
| | | | (SEQ ID NO. 36) |
| rs2239681 | Forward | Forward | 5′-GTTGGAGCTGGAGGCACA-3′ |
| | | | (SEQ ID NO. 37) |
| | | Reverse | 5′-AAATCAGCCTGAAGAGTCACC-3′ |
| | | | (SEQ ID NO. 38) |
| | | Genotyping | 5′-AGCTGGAGGCACATGGATTGGAGTCCCTGTACCTGCC |
| | | | CCA-3′ |
| | | | (SEQ ID NO. 39) |
| rs3802971 | Forward | Forward | 5′-ATCATCTTTGCCCRTCTCC-3′ |
| | | | (SEQ ID NO. 40) |
| | | Reverse | 5′-ACTTCCTACCCCAGAACTCC-3′ |
| | | | (SEQ ID NO. 41) |
| | | Genotyping | 5′-GGGGGCCGTGCACTGATG-3′ |
| | | | (SEQ ID NO. 42) |
| rs3213221 | Reverse | Forward | 5′-CCCTGCAGCTGTGGATGC-3′ |
| | | | (SEQ ID NO. 43) |
| | | Reverse | 5′-CCATGTGCAGAATGAAGC-3′ |
| | | | (SEQ ID NO. 44) |
| | | Genotyping | 5′-GCTGAGCTCCTGCAATAATGACCGTG-3′ |
| | | | (SEQ ID NO. 45) |

1) PCR Amplification

First, a region including a single nucleotide polymorphism was amplified with Multiplex PCR. A composition of PCR reaction solution and condition for PCR reaction used for the PCR reaction are shown in the following Table 3 and Table 4. More specifically, DNA was isolated from a clinical sample and used as a DNA template for the PCR reaction. PCR reaction was performed by performing a pre-denaturation (1 cycle) at approximately 95° C. for approximately 15 minutes; a denaturation at approximately 94° C. for approximately 30 seconds, annealing at approximately 55° C. for approximately 1 minute and 30 seconds, and elongation at approximately 72° C. for approximately 1 minute and 30 seconds as 1 cycle (35 cycles); and finally a final elongation at approximately 72° C. for approximately 10 minutes. The reactant was stored at approximately 4° C. In the above-described PCR reaction, the forward and reverse primers to each of single nucleotide polymorphisms in Table 2 described above were used as a primer for PCR reaction.

TABLE 3

| Reagent | Volume (Amount per 1 well) (μl) |
|---|---|
| 10X buffer | 1 |
| MgCl₂ (25 mM) | 1.4 |
| dNTP (10 mM) | 0.3 |
| primer pool (100 pmol/μl) | 0.24 |
| Taq (5 U/μl) | 0.2 |
| Distilled Water (DW) | 5.86 |
| DNA | 1 |
| Total | 10 |

TABLE 4

| Temperature | Time | Cycle |
|---|---|---|
| 95° C. | 15 minutes | 1 |
| 94° C. | 30 seconds | 35 |
| 55° C. | 1 minute and 30 seconds | |
| 72° C. | 1 minute and 30 seconds | |
| 72° C. | 10 minutes | 1 |
| 4° C. | ∞ | — |

2) PCR Product Purification: SAP & Exo I Treatment (10 μl)

In order to complete PCR reaction (SNaPshot reaction) for a primer extension, the PCR product was purified. That is, the following SAP & Exo I was treated to the PCR product. The composition of purified reaction materials and reaction condition of the product are shown in the following Table 5 and Table 6, respectively.

TABLE 5

| Material | Volume (μl) |
|---|---|
| SAP (1 unit/μl) | 5 |
| Exo I (10 unit/μl) | 0.2 |
| PCR Product | 4 |
| Distilled Water | 0.8 |
| Total | 10 |

TABLE 6

| Reaction Temperature | Reaction Time |
|---|---|
| 37° C. | 1 hour |
| 72° C. | 15 minutes |

3) SNaPshot Reaction: PCR Reaction for One Base Extension

SNaPshot Reaction Premix (Applied Biosystems, CA, USA) and genotyping primer to each of single nucleotide polymorphisms in the above Table 2 were mixed with the purified PCR product, and then PCR reaction was performed. The composition of SNaPshot reaction materials and reaction condition are shown in the following Table 7 and Table 8, respectively. The PCR reaction was performed by performing a denaturation at approximately 96° C. for approximately 10 seconds, an annealing at approximately 50° C. for approximately 5 seconds, and an elongation at approximately 60° C. for approximately 30 seconds as 1 cycle (25 cycles).

TABLE 7

| Material | Volume (μl) |
|---|---|
| SNaPshot Ready Reaction Premix | 5 |
| Genotyping primer pool (0.15 pmol/μl) | 1 |
| Purified PCR Product | 2 |
| Distilled Water | 2 |
| Total | 10 |

TABLE 8

| Reaction Temperature | Reaction Time |
|---|---|
| 96° C. | 10 seconds |
| 50° C. | 5 seconds |
| 60° C. | 30 seconds |
| 25 cycle | — |

4) SAP Treatment: Process for Removing Non-reacted Oligonucleotide

In order to remove a non-reacted oligonucleotide, SAP was added and treated to the SNaPshot reaction product. The composition of the SAP treatment reaction materials and the reaction condition are shown in the following Table 9 and Table 10, respectively.

TABLE 9

| Material | Volume (μl) |
|---|---|
| SAP (1 unit/μl) | 1 |
| Distilled Water | 1 |

TABLE 10

| Reaction Temperature | Reaction Time |
|---|---|
| 37° C. | 1 hour |
| 72° C. | 15 minutes |

5) Running

An analysis was performed by using an automatic sequencer, such as ABI 3730XL (Applied Biosystems, CA, USA). At this time, the nucleotide sequence at a single nucleotide polymorphism (SNP) position was determined as a fluorescent color of analysis result.

6) Data Analysis

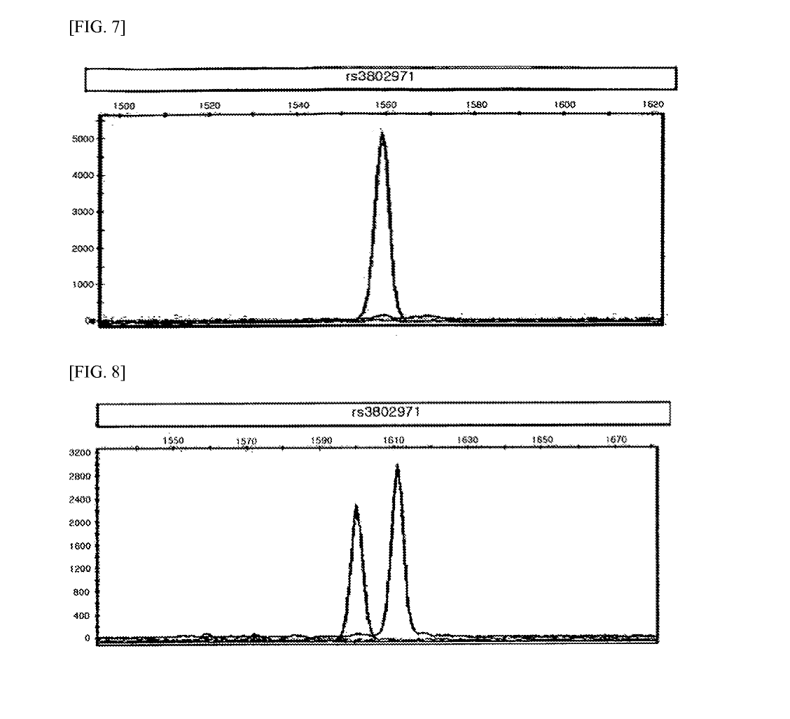

By analyzing the product subjected to the one base extension through ABI 3730XL that is an automatic sequencer, only the labeled part was detected to exhibit a peak, as illustrated in FIGS. 6 to 8. The sequence of the single nucleotide polymorphism could be confirmed by analyzing the fluorescent color exhibited as the peak (each of the bases used ddNTP labeled with the fluorescent dye with different colors each other).

When using a test kit for predicting recurrence of hepatocellular carcinoma and a method of analyzing single nucleotide polymorphisms for predicting recurrence of hepatocellular carcinoma according to an embodiment (Example 3) of the present invention, the single nucleotide polymorphisms of rs3741208, rs2585, rs2239681, and rs3213221 could be multiplexly analyzed. However, rs3802971 that is a single nucleotide polymorphism exhibiting a significant correlation with recurrence of hepatocellular carcinoma after curative surgical resection could not be multiplexly analyzed, and was needed to be a separate analysis.

According to the present invention, a micro-array or a test kit for predicting recurrence of hepatocellular carcinoma can be developed by using a single nucleotide polymorphism (SNP) that is useful for predicting recurrence of hepatocellular carcinoma after curative surgical resection of hepatocellular carcinoma, and the postoperative recurrence rate of hepatocellular carcinoma can be decreased by screening a drug for reducing recurrence of hepatocellular carcinoma.

1. A single nucleotide polymorphism (SNP) for predicting recurrence of hepatocellular carcinoma, the SNP comprising at least one polynucleotide selected from the group consisting of C allele (CC or GC genotype) in −291C/G (rs3213221) of IGF2 gene; T allele (CT or TT genotype) in −13021C/T (rs3741208) of IGF2 gene; T allele (CT or TT genotype) in 66378C/T (rs1048201) of FGF2 gene; G allele (GG or GA genotype) in 50012A/G (rs6534367) of FGF2 gene; GC haplotype in 6310 (rs2585)/4702 (rs3802971) of IGF2 gene; homozygotic CC haplotype in −11228 (rs2239681)/−13021 (rs3741208) of IGF2 gene; and CT haplotype in −11228 (rs2239681)/−13021 (rs3741208) of IGF2 gene; or a complementary nucleotide thereof.

2. The single nucleotide polymorphism (SNP) for predicting the recurrence of hepatocellular carcinoma according to claim 1, wherein the recurrence of hepatocellular carcinoma is recurrence of hepatocellular carcinoma in patients treated with curative surgical resection.

3. A test kit for predicting recurrence of hepatocellular carcinoma, the test kit using a single-base extension reaction and comprising:
a forward primer for amplifying −13021 (rs3741208) region of IGF2 gene;
a reverse primer for amplifying −13021 (rs3741208) of IGF2 gene;
a primer for genotyping −13021 (rs3741208) region of IGF2 gene;
a forward primer for amplifying 6310 (rs2585) region of IGF2 gene;
a reverse primer for amplifying 6310 (rs2585) region of IGF2 gene;
a primer for genotyping 6310 (rs2585) region of IGF2 gene;
a forward primer for amplifying −11228 (rs2239681) region of IGF2 gene;
a reverse primer for amplifying −11228 (rs2239681) region of IGF2 gene;
a primer for genotyping −11228 (rs2239681) region of IGF2 gene;
a forward primer for amplifying 4702 (rs3802971) region of IGF2 gene;
a reverse primer for amplifying 4702 (rs3802971) region of IGF2 gene;
a primer for genotyping 4702 (rs3802971) region of IGF2 gene;
a forward primer for amplifying −291C/G (rs3213221) region of IGF2 gene;
a reverse primer for amplifying −291C/G (rs3213221) region of IGF2 gene; and
a primer for genotyping −291C/G (rs3213221) region of IGF2 gene.

4. The test kit for predicting recurrence of hepatocellular carcinoma according to claim 3, wherein the forward primer for amplifying −13021 (rs3741208) region of IGF2 gene is a primer of SEQ ID NO. 17;
the reverse primer for amplifying −13021 (rs3741208) of IGF2 gene is a primer of SEQ ID NO. 18;
the primer for genotyping −13021 (rs3741208) region of IGF2 gene is a primer of SEQ ID NO. 35;
the forward primer for amplifying 6310 (rs2585) region of IGF2 gene is a primer of SEQ ID NO. 20;
the reverse primer for amplifying 6310 (rs2585) region of IGF2 gene is a primer of SEQ ID NO. 21;
the primer for genotyping 6310 (rs2585) region of IGF2 gene is a primer of SEQ ID NO. 36;
the forward primer for amplifying −11228 (rs2239681) region of IGF2 gene is a primer of SEQ ID NO. 37;
the reverse primer for amplifying −11228 (rs2239681) region of IGF2 gene is a primer of SEQ ID NO. 38;
the primer for genotyping −11228 (rs2239681) region of IGF2 gene is a primer of SEQ ID NO. 39;
the forward primer for amplifying 4702 (rs3802971) region of IGF2 gene is a primer of SEQ ID NO. 40;
the reverse primer for amplifying 4702 (rs3802971) region of IGF2 gene is a primer of SEQ ID NO. 41;
the primer for genotyping 4702 (rs3802971) region of IGF2 gene is a primer of SEQ ID NO. 42;
the forward primer for amplifying −291C/G (rs3213221) region of IGF2 gene is a primer of SEQ ID NO. 43;
the reverse primer for amplifying −291C/G (rs3213221) region of IGF2 gene is a primer of SEQ ID NO. 44; and
the primer for genotyping −291C/G (rs3213221) region of IGF2 gene is a primer of SEQ ID NO. 45.

5. A method for predicting recurrence of hepatocellular carcinoma, the method comprising:
a step of obtaining a nucleic acid sample from a clinical specimen; and
a step of determining a nucleotide sequence of at least any one polymorphism regions of at least one polynucleotide selected from the group consisting of C allele (CC or GC genotype) in −291C/G (rs3213221) of IGF2 gene; T allele (CT or TT genotype) in −13021C/T (rs3741208) of IGF2 gene; T allele (CT or TT genotype) in 66378C/T (rs1048201) of FGF2 gene; G allele (GG or GA genotype) in 50012A/G (rs6534367) of FGF2 gene; GC haplotype in 6310 (rs2585)/4702 (rs3802971) of IGF2 gene; homozygotic CC haplotype in −11228 (rs2239681)/−13021 (rs3741208) of IGF2 gene; and CT haplotype in −11228 (rs2239681)/−13021 (rs3741208) of IGF2 gene; or a complementary nucleotide thereof.

6. The method for predicting recurrence of hepatocellular carcinoma according to claim 5, wherein the step of determining the nucleotide sequence of the polymorphism region includes a step of hybridizing the nucleic acid sample to a micro-array fixed with the polynucleotide or the complementary nucleotide thereof and a step of detecting a hybridization result thus obtained.

7. A method for screening a drug to prevent recurrence of hepatocellular carcinoma, the method comprising:
a step of contacting a polypeptide encoded by the polynucleotide or the complementary nucleotide thereof of the single nucleotide polymorphism (SNP) for diagnosing recurrence of hepatocellular carcinoma according to claim 1 with a candidate material; and
a step of determining whether the candidate material has activity to enhance or inhibit a function of the polypeptide.

8. A method for screening a drug to prevent recurrence of hepatocellular carcinoma, the method comprising:
a step of contacting a polypeptide encoded by the polynucleotide or the complementary nucleotide thereof of the single nucleotide polymorphism (SNP) for diagnosing recurrence of hepatocellular carcinoma according to claim 2 with a candidate material; and
a step of determining whether the candidate material has activity to enhance or inhibit a function of the polypeptide.


SEQUENCE LISTING (53 sequences)

SEQ ID NO: 1 - DNA, 52 bp, Homo sapiens
gctgagctcctgcaataatgaccgtgsagatggtcacccctcgtgtaaaatt

SEQ ID NO: 2 - DNA, 52 bp, Homo sapiens
aaattggacaggagactgaggagaaaygccgggagaggcaacaaccgccctc

SEQ ID NO: 3 - DNA, 52 bp, Homo sapiens
aagctccaggatttgtgtgctgttgcygaatactcaggacggacctgaattc

SEQ ID NO: 4 - DNA, 52 bp, Homo sapiens
aataaatgtatacaattttgattattrgtttttaagtttcttctttagaatg

SEQ ID NO: 5 - DNA, 52 bp, Homo sapiens
tgccggaaatattagcgttaaaggagytgagttgagtcaaacacgggccgca

SEQ ID NO: 6 - DNA, 52 bp, Homo sapiens
gagtcctcgggggccgtgcactgatgyggggagtgtgggaagtctggcggtt

SEQ ID NO: 7 - DNA, 52 bp, Homo sapiens
ggattggagtccctgtacctgccccaygacagggcctgcagggagggatcca

SEQ ID NO: 8 - DNA, 25 bp, Artificial Sequence
  Forward primer for rs1048201
atgatatacatatctgacttcccaa

SEQ ID NO: 9 - DNA, 27 bp, Artificial Sequence
  Reverse primer for rs1048201
aagagactggtataaaatcagaattca

SEQ ID NO: 10 - DNA, 45 bp, Artificial Sequence
  Genotyping primer for rs1048201
cgtgccgctcgtgatagaatagctccaggatttgtgtgctgttgc

SEQ ID NO: 11 - DNA, 26 bp, Artificial Sequence
  Forward primer for rs6534367
ttcttctattatgcarttgtttgaag

SEQ ID NO: 12 - DNA, 28 bp, Artificial Sequence
  Reverse primer for rs6534367
ttacattctcaactagtgttctacattg

SEQ ID NO: 13 - DNA, 45 bp, Artificial Sequence
  Genotyping primer for rs6534367
acgcacgtccacggtgatttataaatrtatacaattttgattatt

SEQ ID NO: 14 - DNA, 18 bp, Artificial Sequence
  Forward primer for rs308428
gcatgttttgggaaccaa

SEQ ID NO: 15 - DNA, 22 bp, Artificial Sequence
  Reverse primer for rs308428
attaaaaccctccattgactcc

SEQ ID NO: 16 - DNA, 45 bp, Artificial Sequence
  Genotyping primer for rs308428
cgtgccgctcgtgatagaatgaagtttggaatggcaagaagtaag

SEQ ID NO: 17 - DNA, 19 bp, Artificial Sequence
  Forward primer for rs3741208
acaggtaaagcttccttcc

SEQ ID NO: 18 - DNA, 17 bp, Artificial Sequence
  Reverse primer for rs3741208
gccatcaggaggagaga

SEQ ID NO: 19 - DNA, 45 bp, Artificial Sequence
  Genotyping primer for rs3741208
cgactgtaggtgcgtaactcgagggcvgttgttgcctctcccggy

SEQ ID NO: 20 - DNA, 18 bp, Artificial Sequence
  Forward primer for rs2585
aatgtcacctgtgcctgc

SEQ ID NO: 21 - DNA, 22 bp, Artificial Sequence
  Reverse primer for rs2585
ttaaagacaaaacccaagcatg

SEQ ID NO: 22 - DNA, 25 bp, Artificial Sequence
  Genotyping primer for rs2585
tgcggcccgtgtttgactyaactca

SEQ ID NO: 23 - DNA, 18 bp, Artificial Sequence
  Forward primer for DL1002505
tgtccggcagcaggagga

SEQ ID NO: 24 - DNA, 18 bp, Artificial Sequence
  Reverse primer for DL1002505
acgcacactgccagacca

SEQ ID NO: 25 - DNA, 25 bp, Artificial Sequence
  Genotyping primer for DL1002505
aggactgggcctcctgcgtgctggc

SEQ ID NO: 26 - DNA, 18 bp, Artificial Sequence
  Forward primer for rs308395
gaggcacgtccatacttg

SEQ ID NO: 27 - DNA, 18 bp, Artificial Sequence
  Reverse primer for rs308395
cagcgtctcacacactga

SEQ ID NO: 28 - DNA, 25 bp, Artificial Sequence
  Genotyping primer for rs308395
ctcttctatggcctactttctactg

SEQ ID NO: 29 - DNA, 18 bp, Artificial Sequence
  Forward primer for rs11938826
ttggggaaggctgataat

SEQ ID NO: 30 - DNA, 18 bp, Artificial Sequence
  Reverse primer for rs11938826
gccatatttcggctaaca

SEQ ID NO: 31 - DNA, 27 bp, Artificial Sequence
  Genotyping primer for rs11938826
cccagaaaagagggtacttcacaccag

SEQ ID NO: 32 - DNA, 18 bp, Artificial Sequence
  Forward primer for rs3213221
taggacggaggccaggtc

SEQ ID NO: 33 - DNA, 17 bp, Artificial Sequence
  Reverse primer for rs3213221
aggtgcccctcccaaac

SEQ ID NO: 34 - DNA, 25 bp, Artificial Sequence
  Genotyping primer for rs3213221
aattttacacgagggktgaccatct

SEQ ID NO: 35 - DNA, 46 bp, Artificial Sequence
  Genotyping primer for rs3741208
gcctsctgaccaccagcaagaaattggacaggagacygargagaaa

SEQ ID NO: 36 - DNA, 34 bp, Artificial Sequence
  Genotyping primer for rs2585
ggtccmccttgcggcccgtgtttgactyaactca

SEQ ID NO: 37 - DNA, 18 bp, Artificial Sequence
  Forward primer for rs2239681
gttggagctggaggcaca

SEQ ID NO: 38 - DNA, 21 bp, Artificial Sequence
  Reverse primer for rs2239681
aaatcagcctgaagagtcacc

SEQ ID NO: 39 - DNA, 40 bp, Artificial Sequence
  Genotyping primer for rs2239681
agctggaggcacatggattggagtccctgtacctgcccca

SEQ ID NO: 40 - DNA, 19 bp, Artificial Sequence
  Forward primer for rs3802971
atcatctttgcccrtctcc

SEQ ID NO: 41 - DNA, 20 bp, Artificial Sequence
  Reverse primer for rs3802971
acttcctaccccagaactcc

SEQ ID NO: 42 - DNA, 18 bp, Artificial Sequence
  Genotyping primer for rs3802971
gggggccgtgcactgatg

SEQ ID NO: 43 - DNA, 18 bp, Artificial Sequence
  Forward primer for rs3213221
ccctgcagctgtggatgc

SEQ ID NO: 44 - DNA, 18 bp, Artificial Sequence
  Reverse primer for rs3213221
ccatgtgcagaatgaagc

SEQ ID NO: 45 - DNA, 26 bp, Artificial Sequence
  Genotyping primer for rs3213221
gctgagctcctgcaataatgaccgtg

SEQ ID NO: 46 - DNA, 46 bp, Artificial Sequence
  Genotyping primer for rs3741208
gcctgctgaccaccagcaagaaattggacaggagaccgaagagaaa

SEQ ID NO: 47 - DNA, 46 bp, Artificial Sequence
  Genotyping primer for rs3741208
gcctgctgaccaccagcaagaaattggacaggagactgaagagaaa

SEQ ID NO: 48 - DNA, 46 bp, Artificial Sequence
  Genotyping primer for rs3741208
gcctcctgaccaccagcaagaaattggacaggagaccgaagagaaa

SEQ ID NO: 49 - DNA, 46 bp, Artificial Sequence
  Genotyping primer for rs3741208
gcctcctgaccaccagcaagaaattggacaggagactgaagagaaa

SEQ ID NO: 50 - DNA, 46 bp, Artificial Sequence
  Genotyping primer for rs3741208
gcctgctgaccaccagcaagaaattggacaggagaccgaggagaaa

SEQ ID NO: 51 - DNA, 46 bp, Artificial Sequence
  Genotyping primer for rs3741208
gcctgctgaccaccagcaagaaattggacaggagactgaggagaaa

SEQ ID NO: 52 - DNA, 46 bp, Artificial Sequence
  Genotyping primer for rs3741208
gcctcctgaccaccagcaagaaattggacaggagaccgaggagaaa

SEQ ID NO: 53 - DNA, 46 bp, Artificial Sequence
  Genotyping primer for rs3741208
gcctcctgaccaccagcaagaaattggacaggagactgaggagaaa